(19) United States
(12) Patent Application Publication
(10) Pub. No.: US 2003/0198967 A1
(43) Pub. Date: Oct. 23, 2003

(54) Multi-functional microarrays and methods

(76) Inventors: Robert S. Matson, Orange, CA (US); Raymond C. Milton, La Habra, CA (US); Robert J. Obremski, Yorba Linda, CA (US); John W. Silzel, Yorba Linda, CA (US)

Correspondence Address: JEFFREY G. SHELDON, SHELDON & MAK, 255 SOUTH LAKE AVENUE, 9TH FLOOR, PASADENA, CA, 91101, US

(21) Appl. No.: 10/128,281

(22) Filed: Apr. 23, 2002

(51) Int. Cl. C12Q 1/68; C12P 19/60; C12M 1/34; G01N 33/543; B05D 3/00

(52) U.S. Cl. 435/6; 435/287.2; 436/518; 427/2.11

(57) ABSTRACT

Detection devices for multianalyte detection on a solid substrate, methods for the preparation of the devices and their use in analytical and diagnostic procedures are described. The detection devices include a solid substrate fabricated with an array of detection spots, the detection spots having an analyte sensor bound to the substrate by a universal binding ligand. The universal binding ligand is capable of binding multiple analyte sensors to create a multifunctional array. A process for producing the detection devices and assay methods employing microprinting technology are also described.

### BACKGROUND

The present invention relates to analytical detection devices such as microscopic or miniature chemical or biochemical sensors, probes, and dosimiters, the preparation of spatially resolved analytical regions on these devices, and analytical detection methods employing these devices.

Miniature chemical or biochemical sensor devices have been used in various chemical and biochemical diagnostic and synthetic applications such as: DNA analysis (Southern, E. M., et al., Genomics, 1992, vol. 13, pp. 1008-1017; Pease, A. C., et al., Proc. Natl. Acad. Sci. USA, 1994, vol. 91, pp. 5022-5026; Schena, M., et al, Science, 1995, vol. 270, pp. 467-470; Matson, R. S., et al., Analyt. Biochem, 1995, vol. 224, pp. 110-116); immunoglobulin-based assays (Elkins R., et al., J. Int'l Fed. Clin. Chem., 1997, vol. 9, pp. 100-109); and immuno-diagnostic and screening assays (Mendoza, L. G., et al., BioTechniques, 1999, vol. 27, pp. 778-788; Joos, T. O., Electrophoresis, 2000, vol. 21, pp. 2641-2650). These sensor devices are generally composed of a solid substrate and an analyte specific reagent such as an analyte sensor (e.g., a capture agent). In these systems, analytical samples and reagents are bulk delivered to the sensor. Bulk sample delivery floods the entire surface of the sensor, equally distributing the sample to the sensor. In subsequent processing steps, other reagents such as signal development reagents, and/or rinse reagents are also bulk delivered to the sensor. These sensor devices can be used to process a few samples on the same substrate. However, because the same sample and reagent are delivered to the entire surface of the sensor, analyzing multiple samples with high throughput is an issue in these systems.

Miniature assay systems based on a microtiter plate format employing a single capture agent are also known. These assay systems have not yet overcome the problems associated with small volume delivery such as evaporation and inadequate aspiration and dispense fluidics.

Other miniature assay systems based on individual assay sites are also known (Matson, R. S., et al., Analyt. Biochem, 1994, vol. 217, pp. 306-310). These systems have not yet fully overcome the problems associated with attaching the analyte specific reagents to the solid substrate. Inadequate attachment of the analyte specific reagents to the solid substrate can result in leaching of the reagents into solution. When reagents leach into solution, they will fail to provide a signal, resulting in inaccurate results. Attempts attach the analyte specific reagent to the substrate have included complex and expensive manipulation of the solid substrate; and derivatization each analyte specific reagent with a covalent linking agent to immobilize the capture agent onto the surface of the solid substrate. See, e.g., Silzel, J. W., et al., J. Clin. Res., (1998) 44:2036-2043; Lindmark R., et al. J. Immunological Methods (1983), 62: 1-13; Matson, R. S., et al., J. Chromatography (1988) 458:67-77, and U.S. Pat. No. 6,110,669. For example, arrays have been fabricated by activating the entire surface of a solid substrate with a coupling agent. An analyte specific reagent is then printed, stamped, or otherwise patterned onto the activated solid substrate. The unused coupling agent between patterned zones is then inactivated or passivated to create the array. These systems can be costly because of the waste associated with using large quantities of the coupling agent.

Thus, prior analyte detection systems employing micro sensor technology suffer from one or more of the following disadvantages: 1) reagent waste through bulk sample delivery to the solid substrate; 2) insufficient throughput for multiple sample analysis; 3) insufficient attachment of the analyte specific capture agent to the solid substrate; and 4) inadequate or expensive dispensing and aspiration fluidics. A need, therefore, exists for a high throughput detection device that sufficiently attaches an analyte specific reagent to a substrate where low cost fluid delivery systems that minimize reagent waste are employed.

### SUMMARY

The present invention is for a detection device and methods that satisfies these needs. The analyte detection device employs a substrate having an array of detection spots on the substrate. The detection spots each have an analyte sensor bound to the substrate by one or more than one binding ligand and there are a plurality of different analyte sensors for different analytes and the same binding ligand is used for two or more than two different analyte sensors. The substrate can have pendant acyl fluoride functionalities to immobilize the detection spots on the substrate by covalent bonding of the binding ligand to the pendant acyl fluoride functionalities. Each of the binding ligand and/or the analyte sensor(s) can be applied to the substrate in a predetermined pattern of substantially localized spots by printing.

In a method for detecting analytes according to the present invention, a plurality of different analytes in a sample can be detected by selecting a device as described above and placing an analytical sample onto the substrate substantially only on the detection spots.

In another method for detecting analytes according to the present invention, an analytical device can be selected, where the analytical device is comprised of a substrate and an array of detection spots on the substrate. In this analytical device, each detection spot is comprised of an analyte sensor and a binding ligand. According to this method, a plurality, or more than one, analytical samples are printed substantially only on each of the detection spots.

The present invention also provides for methods for preparing detection devices and methods for detecting analytes. In these methods, one, or more than one, or all of: the binding ligand, the analyte sensor, the analytical sample and/or subsequent processing reagents can be printed onto the substrate. In one method, an analytical sample is placed substantially only on the detection spots by printing the analytical sample onto the detection spots. Subsequent processing steps such as washing and applying signal developing reagents can also be performed by printing the reagents onto the substrate. In another method, an analyte detection device is prepared by printing a plurality of analyte sensors are on an array of binding ligand spots. In this method, each analyte sensors is for a different analyte and the same binding ligand is used for two, or more than two different analyte sensors. In another method for detecting analytes in a sample, an analytical sample is placed substantially only on an analyte sensor spot by printing. In this method, the array of analyte sensor spots comprises two or more than two different analyte sensors. Further according to this method, detection labels and other processing and development reagents can also be printed on the analyte sensor spots.

The present invention also provides for a multi-step synthetic method where a substrate is selected and an array of binding ligand spots is placed on the substrate. One or more than one synthetic reagents are placed on the array of binding ligand spots. In this method, there are a plurality of different reagents for different syntheses, the same binding ligand is used for two or more than two different syntheses, and the synthetic reagents are placed substantially only on each binding ligand spot by printing.

### DRAWINGS

These and other features, aspects and advantages of the present invention will become better understood with regard to the following description, appended claims, and accompanying figures where:

FIG. 1 schematically shows the preparation of a detection device according to the present invention.

FIG. 2 schematically shows a preferred method for preparing a universal microrarray according to the present invention.

FIG. 3 illustrates an assay system according to the present invention employing Protein A as a universal binding ligand in an array.

FIG. 4 illustrates a microarray according to the present invention employing an avidin-biotin complex.

FIG. 5 is a flow chart illustrating the steps of a method according to the present invention.

FIG. 6 illustrates a Protein A microarray according to the present invention with overprinting of antibodies.

FIG. 7 illustrates an immunoassay according to the present invention employing printing technology.

FIG. 8 graphically shows the results of the immunoassay illustrated in FIG. 7.

FIG. 9 graphically shows the results of titration of antigen at reduced antibody loading as illustrated in FIG. 7.

FIG. 10 graphically shows the results of a determination of the LLD for reduced capture antibody loading as illustrated in FIG. 7.

### DESCRIPTION

The present invention is adaptable to those applications that include a patterned immobilization of analytical reagents, sensors, or other biological or chemical materials on a solid substrate for further reaction, binding, complexing, or sensing of biological or chemical materials. Examples of systems adaptable to the present invention include various array based clinical assay systems and solid phase synthetic chemistry systems. The present invention can be used in clinical analysis and research for identifying drugs of abuse infectious disease, and blood analytes, drug discovery, structure-functional research, forensics, environmental testing, chemical exposure dosimetry, chemical synthesis, oligoneucleotide and peptide synthesis, combinatorial library creation, cell-based assays, etc.

According to the present invention, a universal binding ligand is attached to a substrate (i.e., a solid support or a solid substrate) in a known pattern. Multiple reactive biological or chemical materials, such as analyte sensors, are subsequently attached to the universal binding ligand to create a template or an array. The template or array can then be further reacted with an analytical sample (in the case of assay systems) for multiplexed analysis, or other biological or chemical materials (for synthetic chemical applications).

Employing a universal binding ligand permits attachment of the reactive chemical or biological materials, such as analyte sensors, to the surface of a substrate without the need to individually derivatize each of the chemical or biological materials so they will adequately attach (i.e., immobilize) onto the substrate.

Employing a universal binding ligand, as described in the present invention, allows commonly available “off-the-shelf” biological or chemical materials, such as derivatized analyte specific reagents, to be used to create analytical and synthetic arrays. Arrays created in this manner can be used in multiplexed high throughput analysis or synthesis. The present invention is of particular use in microscopic or miniature multifunctional chemical or biochemical sensors, probes, dosimeters or other analytical devices.

The universal binding ligand is also referred to as a binding ligand, a universal binding reagent, or a universal linker. The binding ligand is a compound, complex, ligand, or reagent that is capable of attaching or coupling a variety of biological or chemical materials to a solid substrate. The binding ligand can be one of a protein, enzyme, carbohydrate, nucleic acid, oligonucleotide, polynucleotide, aptamer, hapten, drug, dye, small organic molecule, cell, cell fragment, receptor or cell surface binding agent, or their analogs, mimics, conjugates, or composites thereof as known to those of skill in the art with reference to this disclosure. Exemplary binding ligands include anti-ligand proteins such as Protein A, or Protein G, or receptors such as streptavidin. Preferred, but not required universal binding ligands are Protein A and streptavidin. Other binding ligands are cell attachment factors such as fibronectin. Single-component arrays can also be created according to the present invention, preferably using conventional avidin- and biotin-labeled reagents.

In a preferred, but not required embodiment of the present invention, the binding ligand is immobilized directly to a substrate by covalent attachment of the binding ligand to the substrate. The universal binding ligand can be covalently attached to the substrate by activating (i.e., derivatize) the substrate. The substrate can be activated by heat, radiation, or chemical techniques known to those skilled in the art.

Substrates useful in the present invention, also referred to in the art as solid supports, and solid substrates are porous or non-porous materials capable of supporting the binding ligand and the corresponding analytical or synthetic array. Substrates useful in the present invention are known and the application substrate technology to the present invention will be understood by those of skill in the art with reference to this disclosure. For example, substrates can be fabricated from, including, but not limited to polymeric materials, glasses, ceramics, gels, membranes, natural fibers, silicons, metals and composites thereof. The solid substrate can be fabricated in a variety of shapes and sizes depending on the particular use. Examples include plates, sheets, films, and threads. Preferred, but not required shapes are those with flat planar surfaces, such as a microplate, that can be handled by an automated diagnostic system.

In a preferred, but not required aspect of the invention, the substrate is activated by fabricating the substrate from a polymeric material having at least one surface with attached acyl fluoride functionalities. Substrates with derivatized acyl fluoride functionalities can be prepared from a wide range of polymeric materials including those with pendant carboxyl functionalities or those capable of modification to support carboxyl groups that are in turn capable of reaction with suitable reagents to form acyl fluoride functionalities. A description of solid substrates fabricated from polymeric materials with pendant acyl fluoride functionalities is contained in U.S. Pat. No. 6,110,669, incorporated herein by reference. Activated substrates can also be prepared by coating an inert solid substrate with a polymer having attached acyl fluoride functionalities. Other covalent attachment chemistries are also applicable, but not limited to, anhydrides, epoxides, aldehydes, hydrazides, acyl azides, aryl azides, diazo compounds, benzophenones, carbodiimides, imidoesters, isothiocyanates, NHS esters, CNBr, maleimides, tosylates, tresyl chloride, maleic anhydrides and carbonyldiimidazoles. Attachment by non-covalent means or other adsorption mechanisms are also applicable so long as the binding ligand remains attached to the solid support and is capable of binding the analyte sensor.

According to the present invention, a wide variety of customized arrays or templates can be prepared by coupling biological or chemical materials to the universal binding ligand array. The terms “biological materials” and “chemical materials” as used herein, include but are not limited to biological or chemical compounds, complexes, ligands, cells and analytical reagents such as an analyte sensor. An advantage of attaching biological or chemical materials to a universal binding ligand, which is coupled to the solid substrate, is that leaching of the materials is reduced or eliminated.

Array based assay systems for identifying biological analytes typically involve the reaction of analyte-specific biological recognition molecules with an analytical sample. The analyte-specific biological recognition molecule interacts with an analyte of interest and a reporter molecule such as a fluorescent detection dye that can be used to detect the analyte of interest. Biological recognition molecules, are also referred to herein and in the art as analyte sensors, receptors, capture ligands, capture molecules, capture agents and analytical reactants. For purposes of the present invention, an analyte sensor is a chemical or biochemical molecule that can recognize a target analyte and react or bind to the target analyte. The term analyte sensor, as used herein, includes, but is not limited to ions, enzymes, DNA fragments, antibodies, antigens, ligands, haptens, and other biomolecules. For example, when the target analyte is polynucleotide, the analyte sensor can be a polynucleotide that is complementary to the target analyte. When the target analyte is a receptor or a ligand, the analyte sensor can be a ligand or receptor that respectively recognizes the target analyte. An analyte sensor can also be a fluorescent reporter molecule capable of reacting with an analyte, or a specific binding pair member for detecting specific microorganisms and cells such as viruses, fungi, animal and mammalian cells or fragments. Another example of an analyte sensor is a monoclonal antibody, which serves as an antibody catcher. In this example, an epitope recognized by the antibody is bound followed by labeled antibodies specific to the epitope. In still another example, the target analyte may be a drug which is delivered to an immobilized cell that serves as the analyte sensor.

In accordance with embodiments of the present invention, both the target analyte and analyte sensor can be labeled with a reporter molecule. Examples of reporter molecules include but are not limited to, dyes, chemiluminescent compounds, enzymes, fluorescent compounds, metal complexes, magnetic particles, biotin, haptens, radio frequency transmitters, and radioluminescent compounds. One skilled in the art can readily determine the type of reporter molecule to be used to detect a particular target analyte with reference to this disclosure.

In a preferred, but not required aspect of the present invention, an analyte sensor is immobilized on a solid substrate surface by coupling the analyte sensor to a universal binding ligand. An analyte sensor bound to a substrate by a binding ligand is referred to herein as a detection spot.

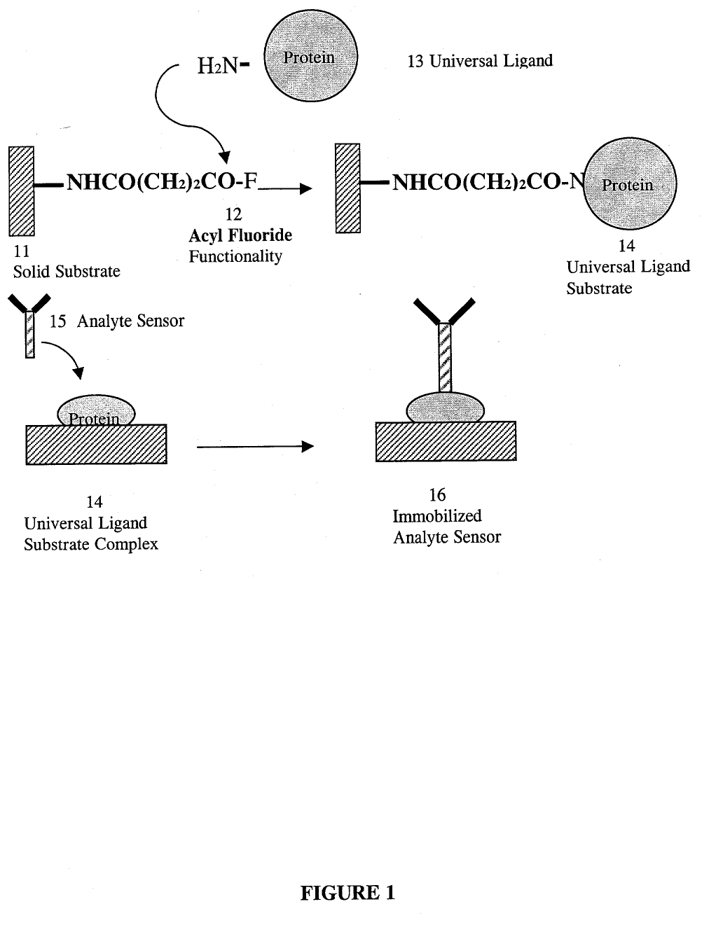

FIG. 1 shows the preparation of a detection device according to the present invention. As shown in FIG. 1, a substrate 11 is shown with an acyl fluoride functionality (CO—F) 12. A universal binding ligand (shown as Protein A) 13 reacts with the acyl fluoride functionality, thereby covalently attaching the universal binding ligand to the substrate 14. An analyte sensor 15 is then coupled to the universal binding ligand substrate complex, to immobilize the analyte sensor on the substrate 11.

Universal binding ligand arrays, created on activated solid substrates, are particularly useful in microassay systems. Microscopic spots can sensitively detect and quantify analytes in dilute solutions. In a preferred, but not required aspect of the invention, a device comprised of a multi-analyte array, matrix, or template can be created. The multi-analyte array is critical by coupling a single universal binding ligand in an array of spots to an activated solid substrate to create a universal binding ligand array. According to the present invention, analyte-specific sensors can be coupled to the universal binding ligand array to create the array of multi-analyte detection spots. In another aspect of the invention, two or more universal binding ligands can be used to create the array. For purposes of this disclosure, the term “spot” refers to an area, region, site, or zone on the substrate or array device. The number of spots in the array device can be varied depending upon the needs of the assay. An array of is comprised of at least two spots, and can be comprised of as many as 10,000 spots or more. In a preferred, but not required aspect of the invention, the array of detection spots is comprised of 16 to 4800 spots, most preferably from about 100 to 400 spots.

The analyte-specific detection spots can be brought into contact with a complex sample mixture such that tens or hundreds of analytes can be analyzed in a quantitative fashion simultaneously. The array of detection spots can be comprised of either the same binding ligand and multiple analyte sensors, or multiple binding ligands and multiple analyte sensors. In a preferred but not limiting example, the number of assays to perform are in multiples of 96, 384 or 1536 corresponding to the number of wells in commercially available microtiter plates. Alternatively, other microwell plates may be fabricated to meet the needs of the assay for reagent reservoirs.

An advantage of this particular aspect of the invention is that miniature support platforms permit smaller sample sizes and reagent volumes, which can lead to economy of scale and timesavings. In addition, the microarray-based analyzers can achieve comparable or greater sensitivity than conventional macro-assay formats.

The present invention also provides a process for preparation of spatially resolved analyte-sensing spots, immobilized on a film, plate, well, or other solid substrate. An assay process employing the multiple analyte-specific detection arrays is also provided.

In accordance with the present invention, a universal binding ligand array can be created by conventional manual application techniques, known to those of skill in the art with reference to this disclosure. In a preferred, but not required embodiment, microarray printing technology can be used to prepare the universal binding ligand array on a solid substrate. According to this embodiment, a universal binding ligand is immobilized on a solid substrate by printing the universal binding ligand in spots on a solid substrate in a matrix.

A universal binding ligand array can also be created by utilizing thermal inkjet printing techniques to “print” a universal binding ligand on selected solid substrate surface sites in an array pattern. Printing techniques utilizing jet printers and piezoelectric microjet printing techniques are described in U.S. Pat. No. 4,877,745, incorporated herein by reference. The method of patterning used in the invention can be changed within the scope of the invention, including, but not limited to: thermal jet printing, piezo jet printing, stamping, sprays, embossing, and optical microlithography.

Printing technology can also be used in aligned micro-printing to prepare assay test spots and deliver analytes and reagents to these spots as a means to conduct microassays and to conduct chemical reactions in microdroplets. For the purposes of this disclosure, employing printing technology to deliver analytes and reagents to assay test sites is termed “overprinting,” or an “overprint assay.” For example, a Hewlett Packard ThinkJet™ desktop printer, employing conventional bit-map graphical binary commands, can be used to align four different printheads to overprint on a substrate to within 10 microns in both X and Y directions (provided the substrate is not removed from the printer between printing steps). A more sophisticated system can provide indexing which allows removal of the substrate between printing steps. The present invention can also be used to move samples under analysis to particular spots in a quantitative manner.

According to a preferred but not required aspect of the present invention, detection spots (i.e., assay test sites) are created on a substrate by printing an array of universal binding ligand spots on a substrate, followed by overprinting analyte sensors over the universal binding ligand array. Microarrayer positioning can be used with 0.5 to 1 micron precision so that high density arrays of detection spots can be created. Thus, detection spots can be created on an array with about a 10 micron spot diameter. Alternately, in another not required aspect of the invention, it is desirable to use detection spots with a larger diameter, such as with low affinity binding ligands or analyte sensors for example. Accordingly, detection spots can be created on an array with about a 500 micron spot diameter. In a preferred but not required aspect of the invention, the detection spots on an array can be from about 75 microns to about 150 microns in spot diameter.

In an aspect of the invention, one or all of the components of an assay such as the analyte sensor, target analyte and reagents can be delivered to the detection spots by printing techniques. One or more than one analytical reagent can be placed on each of the detection spots using printing technology. The delivery can be accomplished in a parallel manner. For example, a micro-ELISA can be used to site-specifically dispense, in a parallel fashion, all components of an assay such as a capture antibodies, antigens, and reporter molecules to the surface of a solid substrate.

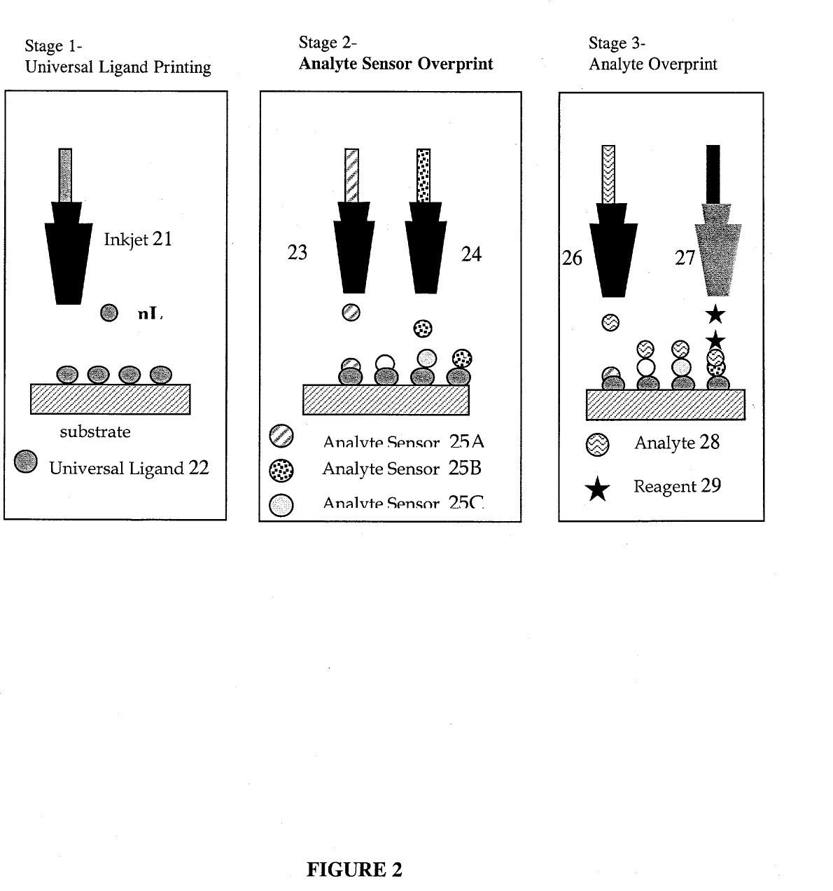

This aspect of the invention is shown schematically in FIG. 2. As shown in FIG. 2, an inkjet printer or similar device dispenses a universal binding ligand and multiple analyte sensors to create an array of assay test sites (i.e., detection spots). As shown in FIG. 2, in stage 1, a universal binding ligand is printed on a substrate. In stage 1, an inkjet printing head 21 dispenses droplets of a universal binding ligand 22. In stage 2, inkjet printing heads 23 and 24 dispense multiple analyte sensors 25A, 25B, and 25C. In stage 3, inkjet printing head 26 dispenses an analytical sample containing an analyte 28 of interest and inkjet printing head 27 dispenses a signal development reagent (i.e., detection reagent) 29.

An advantage of the present invention is that employing an overprint technique, as described herein can result in a 1000-fold reduction in reagent consumption from that used in a conventional 96-well microtiter plate assay. As a further advantage, a level of detection of ˜2 picogram (8×10⁶ molecules per spot) can be achieved at between 4.7 to 37.5 picogram (1.9×10⁷ to 1.5×10⁸ molecules) of capture antibody per spot. Employing the overprint assay technique as described herein, can provide for ultra-low volume sampling. Further, high throughput is achieved by processing arrays in parallel fashion. It is contemplated that future advances in precision printing and environmental control will result in further ultra-low volume sampling and an increased volume of detection spots on smaller substrates.

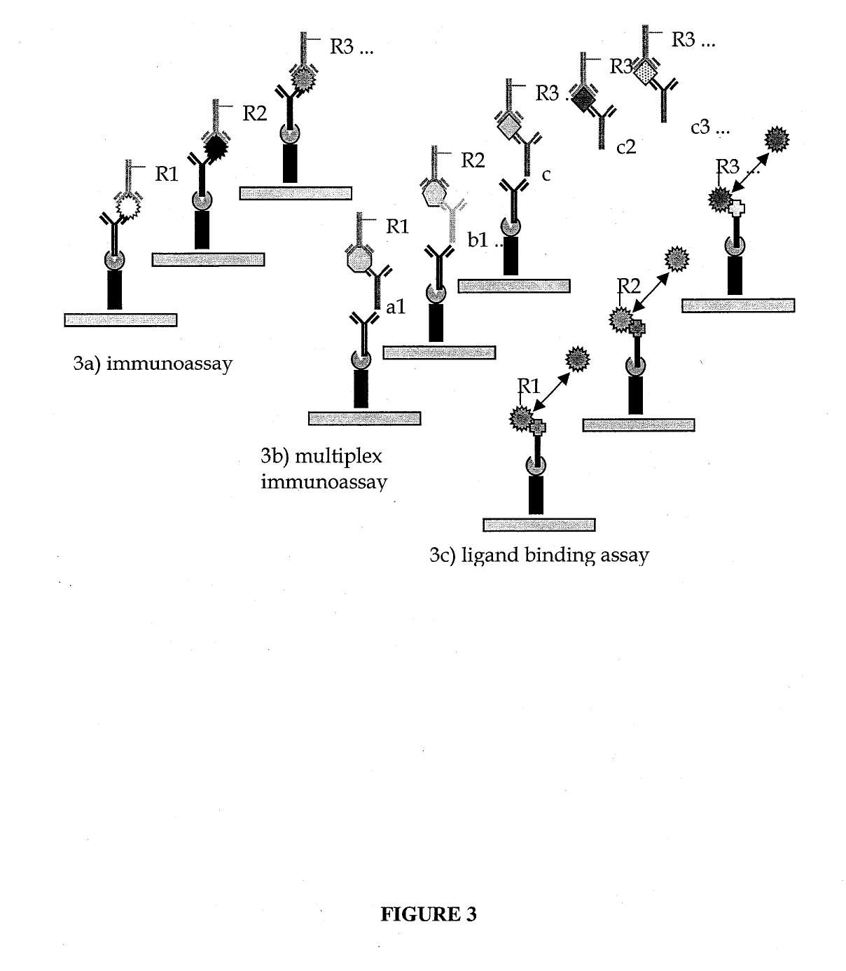

FIG. 3 shows a microassay system, as described herein, employing Protein A as a universal binding ligand to create an array of detection spots for a multiplexed analysis. As shown in FIG. 3, Protein A is used as the universal binding ligand. The universal binding ligand coupled to an activated substrate (shown as an acyl fluoride activated plastic substrate) in a matrix, to create an array of universal binding ligand spots. As shown in FIG. 3, Example 3a), various antibodies can then be delivered to the Protein A spots to create an array of detection spots. The array of antibody detection spots is capable of discriminating between antigens. FIG. 3, Example 3b), shows how the antibody detection spot array is employed in a multiplexed immunoassay. In FIG. 3, Example 3b), different rabbit antibodies which recognize different goat antibodies, which in turn recognize a host of antigens or ligands are used thus achieving a complex immunoassay system. FIG. 3, Example 3c) shows a ligand binding assay. When Protein A is used as the universal binding ligand, it preferentially and reversibly binds to the Fc region of immunoglobulins. See, e.g., Langone, J. J., J. Immunological Methods, 1982, vol. 55, pp. 277-296. Anti-ligand antibodies or Fc-ligand conjugates can be prepared that bind to the Protein A array to create custom ligand assays. In FIG. 3, Example c), the antibody is reduced to its Fc moiety which is in turn conjugated to a series of receptors that can be used in a receptor binding assay. Following analyte, the captured Fc or antibody can be released under acid conditions and the Protein A array regenerated for additional usage.

In a preferred, but not required aspect of the invention, Protein A arrays are created by printing Protein A spots on a substrate. Additional elements of the array can be constructed by micro-dispensing reagents at specific Protein A spots by printing. In a preferred but not required aspect of the present invention, Protein A is prepared in a basic pH buffer system. For jet printing, a LiCl, pH 9-10 buffer solution is preferred. For manual or contact printing, a sodium bicarbonate-carbonate, pH 9-10 buffer solution is preferred. In a preferred, but not required process for jet printing, Protein A is dissolved in an aqueous buffer solution and dispensed in droplets onto an acyl fluoride activated molded ethylene methacrylic acid copolymer substrate. The printed substrate is dried overnight at room temperature. Residual reactive groups are then blocked, for example, by soaking in a casein protein solution for 1 hour, followed by rinsing in distilled water. The array of Protein A binding ligand spots can then be air dried and stored at room temperature.

Streptavidin can also be used to create an array of universal binding ligand spots for a multiplexed analysis. In this aspect of the invention, streptavidin is printed on the substrate to create an array of binding ligand spots. The streptavidin binding ligand array is then reacted with a complementary labeled reagent (i.e., an analyte sensor), specific to the streptavidin binding ligand array to create an array of detection spots for a mutiplexed assay. An example of a complementary labeled reagent is a biotinylated antibody.

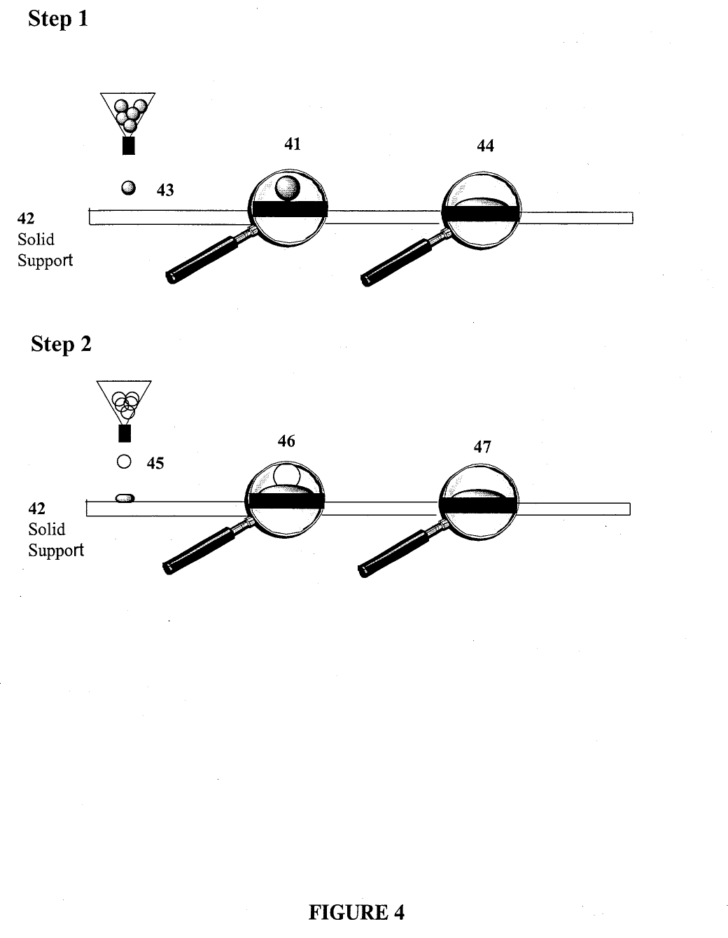

Avidin can also be used as a universal binding ligand to create an array. As shown in FIG. 4, avidin is coupled to a substrate in a matrix to create an avidin universal binding ligand array. In a first step in FIG. 4, an avidin spot 41 is printed on a substrate 42 with an inkjet printer 43. A photoactive coupling agent (e.g., “PhotoLink,” commercially available from Surmodics, Inc.) can be used to immobilize the avidin onto the substrate. In an embodiment of the invention, after printing, the avidin spot containing the coupling agent, is irradiated with UV light, which triggers the formation of covalent bonds with avidin to the substrate. The present invention permits the irradiation, and subsequent immobilization of the binding ligand 44to the substrate, to be conducted prior to the coupling of specific biological materials (i.e., analyte sensors). This is shown, for example, at step (2) in FIG. 4. The pre-irradiation of the universal binding ligand can reduce UV-induced damage to the biological analyte sensors in critical applications.

In a second step, as shown in FIG. 4, which may or may not directly follow the first step, a second printhead 45 can be filled with an analyte specific biotinylated sensing reagent 46 (i.e., a “biotinylated analyte sensor). Examples of these analyte sensors include biotinylated monoclonal mouse anti-human IgG3 or IgG4. In a preferred, but not required aspect of the invention, the biotinylated analyte sensor 46 is brought into alignment with a previously dispensed avidin spot 41. The hydrophilic nature of the avidin-linker residue at this location pulls the printed biotinylated analyte sensor 46 onto the existing avidin 41 spot. The avidin 41 and biotinylated analyte sensor 46 mix and react, prior to drying, to form a product 47 which then dries on the solid substrate 42. The biotinylated detection spot is bound to avidin on the substrate and can be used to perform quantitative assays of IgG3 or IgG4. The process can be repeated, printing additional biotinylated analyte sensors on avidin 41 spots to print an array of detection spots.

The overprinting method described herein is superior to many of the conventional alternatives since the total surface area of the substrate can be orders of magnitude larger than the area actually labeled with analyte sensors. Further, problems associated with activating the entire surface of substrate such as nonspecific binding can be avoided with the devices and methods described herein. Activating the entire surface of a substrate requires passivation of unused sites. Passivation itself can be undesirable since it adds a further step, thereby increasing the cost and time associated with the array fabrication. Also, the surface characteristics of the passivated coupling material must be carefully studied for optimal results.

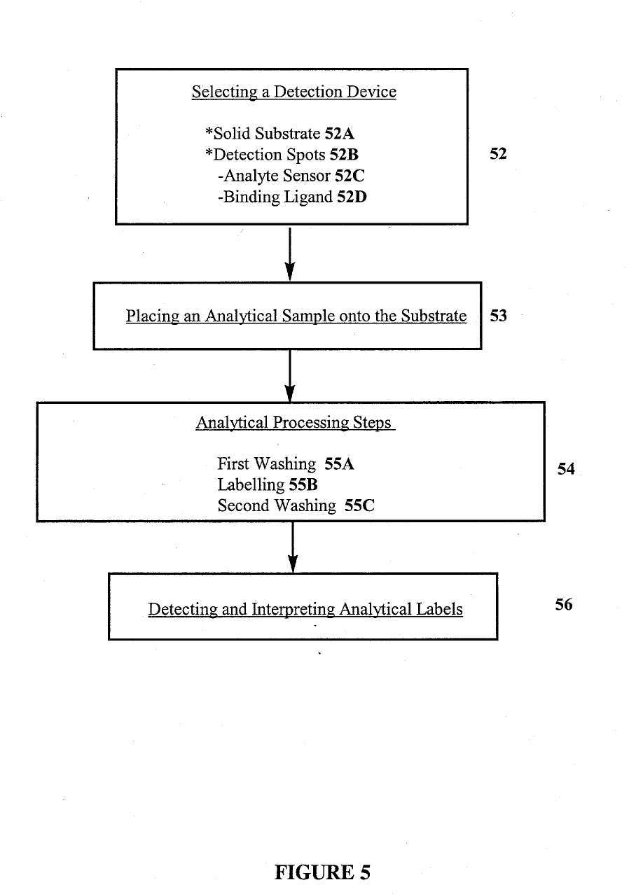

Another aspect of the present invention includes a method for detecting a plurality of different target analytes in a sample. With reference to FIG. 5, a method according to the present invention comprises a first preprocessing stage. In the first preprocessing stage, a detection device 52 for detecting a plurality of analytes is selected, the detection device comprising a solid substrate 52A and an array of detection spots 52B on the substrate. Each detection spot comprises an analyte sensor 52C immobilized on the substrate by a binding ligand 52D.

In a second analytical stage, an analytical sample is placed onto the substrate 53. In a preferred, but not required aspect of the invention, the sample can be printed onto the substrate in discrete droplets, by printing techniques that will be understood to those of skill in the art with reference to this disclosure. In another preferred but not required aspect of the invention, the sample is printed on the substrate in discrete droplets or spots, substantially only on the detection spots, such that one droplet of sample does not significantly flow or contact onto an adjacent droplet of sample. Subsequent analytical processing steps 54 can then be performed such as placing washing 55A, 55C and labelling reagents 55B on the substrate. In a preferred, but not required aspect of the invention, the washing and labelling reagents applied in the processing steps are also printed substantially only on the detection spots.

In a third detection and interpretation stage 56, the presence or absence of an analyte can be detected by determining the presence or absence, respectively of a detection label bound, complexed, or associated with the analyte of interest. Methods for detecting analytical labels and interpretation of the detection results are known and will be understood by those of skill in the art with reference to this disclosure. Examples of detection methods include, but are not limited to fluorescence, phosphorescence, UV, radiolabeling, and the like.

### EXAMPLES

### Example 1

### Preparation of Activated Substrates

The preparation of an activated substrate (i.e., a plastic substrate) in accordance with the present invention is demonstrated in Example 1.

(Diethylamino) sulphur trifluoride (DAST) was obtained from SynChem, Inc. (Aurora, Ohio) and used without purification. DAST reagent consisted of DAST diluted with dichloromethane to 5% v/v. Ethylene methacrylic acid co-polymer (EMA) was obtained from Dupont, molded into various shapes and converted to the acyl fluoride activated form directly using DAST (12). Polypropylene (PP) sheet, Contour 29 (Goex Corp., Janesville, Wis.), 20 mil thickness, was surface animated using a radio frequency plasma amination process (4). The aminated polypropylene sheet was subsequently converted to the carboxyl form using succinic anhydride. The carboxylated PP was in turn modified to acyl fluoride using the DAST reagent.

### Example 2

### Covalent Coupling of Protein A to an Activated Substrate

The covalent coupling of Protein A to an activated substrate (i.e., a plastic substrate) in accordance with the present invention is demonstrated in Example 2. Protein A and certain antibodies were obtained from Zymed Laboratories. Additional antibodies and antigens were purchased from Sigma-Aldrich. An acyl fluoride activated plastic substrates were prepared from the reaction of DAST with carboxyl or amine truncated thermoplastics: ethylene metacrylic acid copolymer (EMA) or plastic aminated polypropylene as described by Matson, R. S., et al., Analyt. Biochem., 1984, vol. 217, pp. 306-310. Protein A microarrays were created either by non-contact dispensing using a BioDot 3200 Dispenser (Cartesian) or by contact printing using the Biomek® 2000 equipped with a 384 pin HDRT. ELF Reagent (ELF-97 Endogenous Phosphatase Detection Kit; Molecular Probes, Inc.), a fluorescent precipitating substrate for alkaline phosphatase was used for signal development. Digital images were obtained using a CCD camera system (Teleris 2, SpectraSource, Inc.). Excitation light at 350 nm was generated using a UV mineral light with signal emission collected at 520 nm using a 10 nm band pass lens filter. The 16-bit images were analyzed using ImaGene software (BioDiscovery, Inc.) then exported as 8-bit values into an Excel spreadsheet (Microsoft) for calculation and graphic display.

Protein A was coupled to acyl fluoride activated substrate in a basic pH buffer medium. Specific coupling conditions varied depending upon the method of printing. These are described below.

### Example 3

### Contact Printing Using the HDRT

Protein A previously reconstituted in deionized water at 2.5 mg/mL was further diluted into sodium carbonate-bicarbonate buffer, 1M, pH 9 at 0.5 to 1 mg/mL. The solution was distributed into a 384-well microplate for dispensing. A sheet of acyl fluoride activated polypropylene (20 mil) was attached to the lid of a microtiter plate cover with double sided sticky tape and placed in a Biomek plate holder. Protein A was dispensed to the surface of acyl fluoride polypropylene in a 3×3 sub-array pattern created using standard Bioworks® software. Up to 384 sub-arrays were created on the surface of the activated plastic substrate in this manner within the 9 cm×12 cm area. Each pin of the dispenser delivered 2-3 nL with a total of 5 dispensings to each site (10 to 15 nL of Protein A solution). The array remained attached to the microplate lid throughout the assay in order to maintain proper indexing on the Biomek worksurface. The Protein A microarray was then blocked in casein (1 mg/mL casein in 50 mM carbonate buffer, 0.15M NaCl, pH 8.5) for 1 hour at room temperature to reduce non-specific adsorption. A final rinse in carbonate buffer followed.

### Example 4

### Non-Contact Printing of Protein A

Non-contact printing of Protein A on a substrate is demonstrated by Example 4.

Protein A at ˜1 mg/mL was dissolved in a 1M LiCl solution at a pH 10 for jet printing onto a substrate. The LiCl solution was used as a carrier in order to maintain droplets on the EMA surface, which was more hydrophilic than the polypropylene substrate. The LiCl/Protein A solution was filtered through a 0.45 Φm Z-Spin Plus™ centrifugal filter to remove protein aggregates. Approximately 16 nL droplets were dispensed onto the molded acyl fluoride activated ethylene methacrylic acid substrates (1 cm×1 cm area). The Cartesian 3200 BioDot Dispenser was used to place droplets of protein A solution on the surface in a 9×9 array pattern at approximately 300 micron center to center spacing. The process of printing Protein A was repeated for a total of 2 to 5 overprints. The subsequent printings were precisely registered to the same spot locations as directed by the user software interface. After printing, the microarrays were removed from the dispenser platform and transferred into a humidity chamber for a 1 hour incubation at 25EC. The microarrays were then placed in a desiccator. After overnight drying at room temperature, the residual reactive groups were blocked by soaking the microarrays in a casein solution (2 mg/mL casein in 50 mM carbonate-bicarbonate buffer, 0.15M NaCl, pH 8.5) for 1 hour. Following a brief rinse in deionized water, the microarrays were air dried for 30 minutes at 25 EC and then stored at room temperature.

### Example 5

### Overprinting Assay

The general process of overprinting is illustrated in FIG. 2. Following the preparation of the Protein A microarrays (stage 1), a series of antibodies were delivered to individual sites (stage 2). Following a rinse to remove unbound capture antibody; antigens were delivered to the array and processed in the same manner (stage 3). In the final step (stage 4) the signal developing reagents were deposited at individual sites of the array. This completes the overnight process. The microarray was then removed from the print stage and signal was read using a CCD camera system.

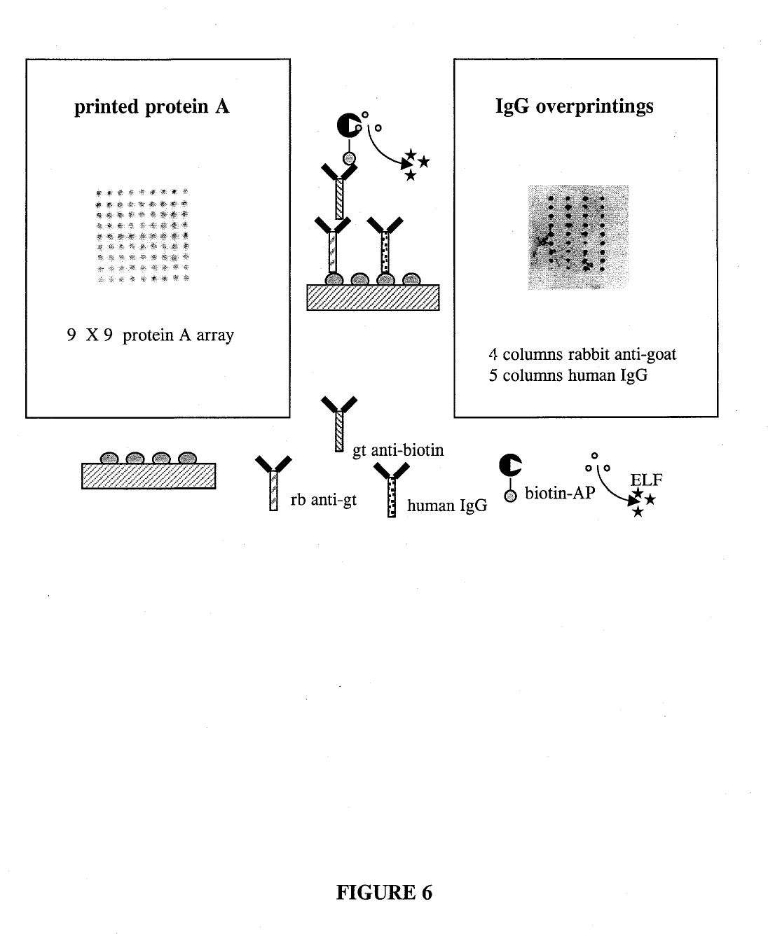

A first experiment is illustrated in FIG. 6. A 9×9 Protein A microarray was created on an EMA molded part and repositioned on a pegboard mounted onto the worksurface of a BioDot dispenser. Capture antibodies (i.e., analyte sensors) were prepared at 1 mg/mL in 50 mM carbonate buffer, 0.1% Tween 20, pH 8.5 and distributed to the wells of a 384-well microtiter plate. Different antibody solutions were dispensed over the elements of the array. The 9×9 Protein A microarray was overprinted with alternate column dispensing of either a rabbit anti-goat IgG or human IgG. In this manner, 4 columns of rabbit immunoglobulin and 5 columns of human immunoglobulin were generated. The microarrays were then removed and placed in a humidified chamber for 1 hour at 25EC to allow complete binding of the antibodies to the protein A sites. The molded parts were then dipped into wash solution to remove unbound antibody and subsequently returned to the BioDot stage. Antigen (goat anti-biotin IgG) was then dispensed to all columns of the array and incubated in the same manner. Following a brief rinse the entire array was incubated with biotinylated-alkaline phosphatase for 30 minutes, rinsed and the signal developed using the ELF reagent for an additional 30 minutes at room temperature.

### Example 6

### Fully Automated Overprint Assay

Example 5 demonstrates the ability to overprint reagents in a semi-automated format. Example 6 demonstrates a fully automated overprint assay according to the present invention.

Arrays of detection spots were created as described herein. The Biomek®2000 Robotic Workstation was employed to deliver both site-specific and bulk reagents to the arrays by automation. In this example, the arrays remained on the worksurface throughout the process. A 384-HDRT was used to deliver small volumes of reagents to specific sites on the array while a P1000 pipet tool was used to dispense bulk rinse reagents. A Gripper tool was used to blot away excess reagents from the array sheets and to cover the plates during incubation. Analyte (antigen) and reporter antibody were delivered to individual spots on the array using the HDRT, incubated and then bulk rinsed using the P1000 pipet tool. In each case, the optimal volume of reagent delivery was determined and the number of repeat dispenses to each site varied as required. In most instances at least 5-7 repeats were required. At the end of each stage in the process the array was blotted dry using filter paper attached to the inside of a microplate lid. The blotter was picked up by the Gripper tool and placed over the array plate for blotting. This was repeated for each rinse cycle using a fresh blotter to avoid carryover of reagents. Following the overprint of reporter antibody, streptavidin-alkaline phosphatase conjugate was printed down. In the final stage, the developing reagent, ELF-97 was applied. The signal was captured off-line using a CCD camera system.

### Example 7

### Antigen Detection

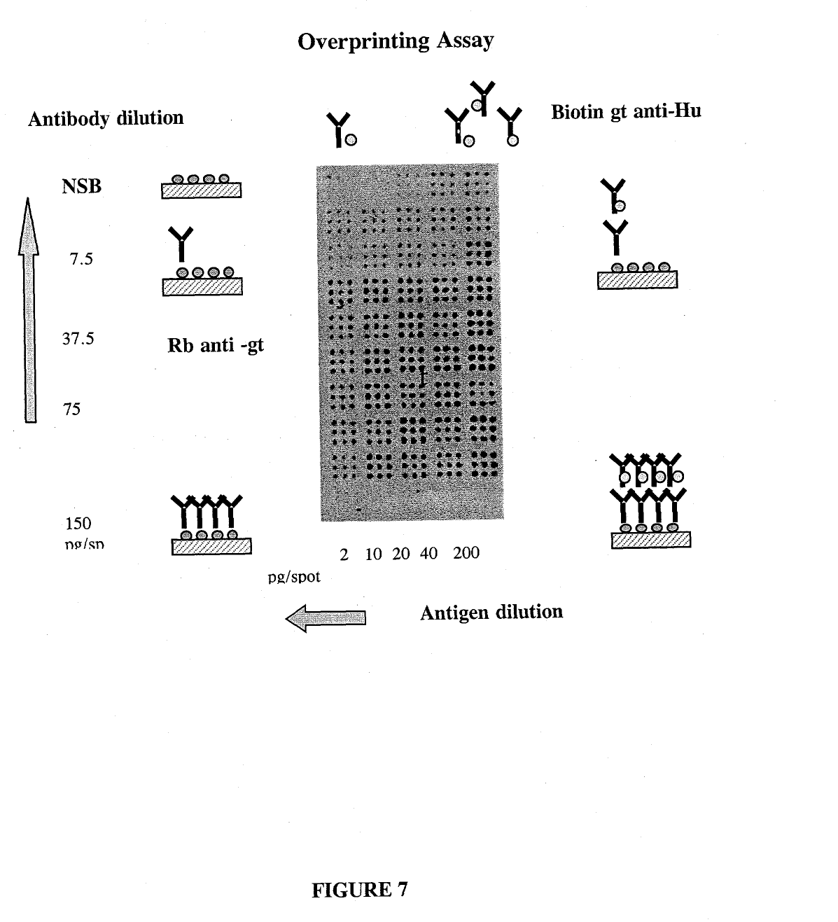

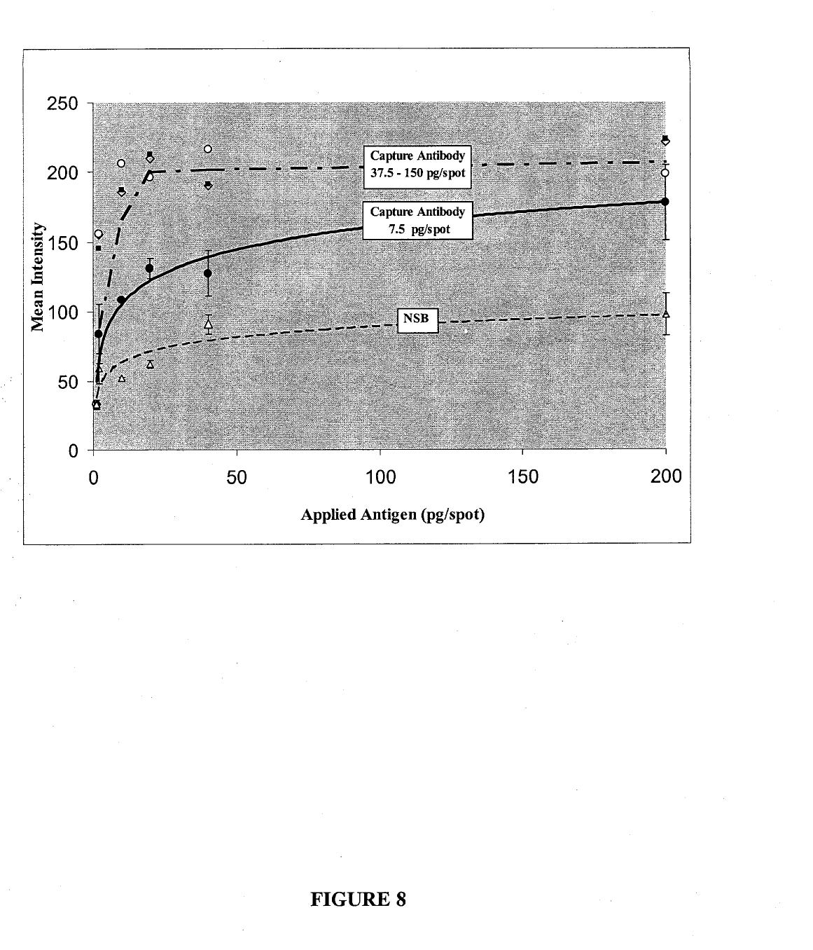

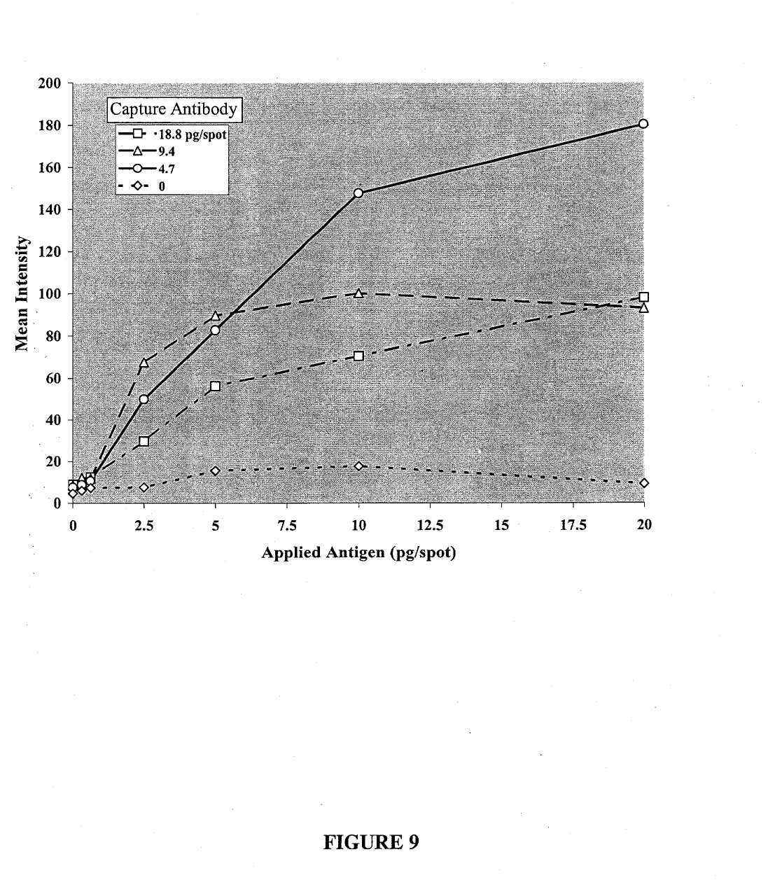

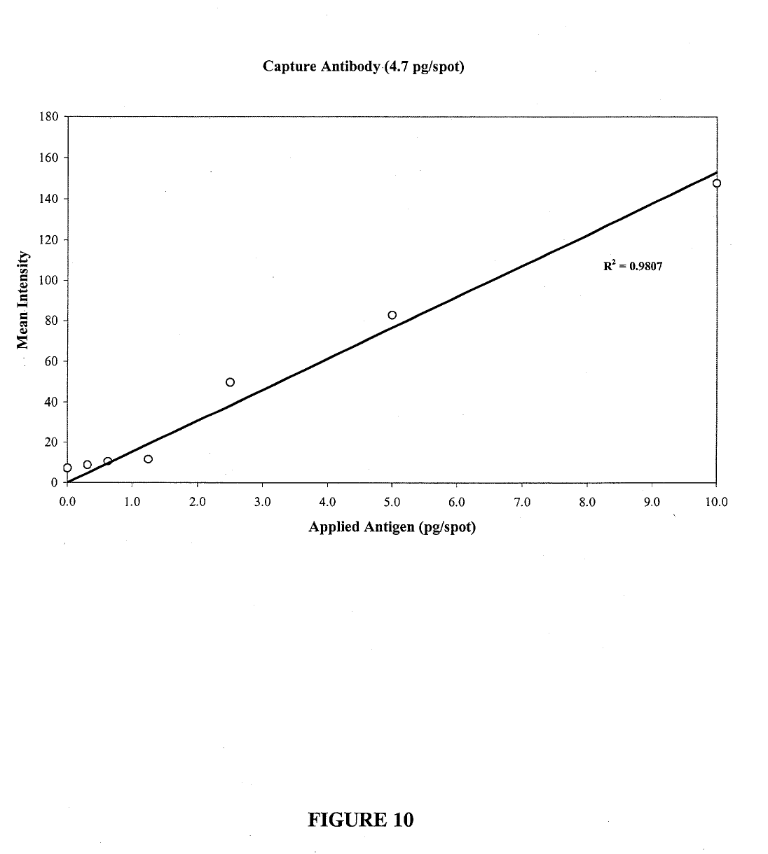

A HDRT was used to print 3×3 sub-arrays (9-replicates) in a 5×9 array of Protein A (stage 1). Next, rabbit anti-goat was overprinted (stage 2) in duplicate at various dilutions from 1:50 (˜150 pg/spot) to 1:1000 (˜7.5 pg/spot) onto the Protein A (3×3) sub-arrays. The top row of Protein A sub-arrays were not overprinted with capture antibody in order to measure the level of non-specific binding of antigen (NSB) at each dilution. Following on-line rising, the antigen (biotin-goat anti-Human antibody, 200 ng/mL) was overprinted (stage 3) onto each sub-array at 1:10 (˜200 pg/spot) to 1:1000 (˜2 pg/spot) v/v dilutions. After a 1 hour incubation the microarray was rinsed and blotted dry as described previously. Next, streptavidin-alkaline phosphatase conjugate was overprinted and each site developed using ELF reagent (stage 4). The resulting image is shown in FIG. 7. A lower level of detection (LLD) for antigen was determined. Based upon a non-competitive immunoassay format (LLD=3 B₀ (SD); where B₀ is the mean background signal and corresponding standard deviations, SD) an antigen sensitivity of 2 pg/spot was achieved, as shown in FIG. 8. This was confirmed from additional experiments in which antigen was serially diluted from 1:800 to 1:6400 v/v in order to achieve antigen samples in the 31 to 250 ng/mL range. Thus, the applied antigen from such solutions would correspond to the delivery of sub-picograms of antigen per capture antibody spot. Likewise, capture antibody was serially diluted to achieve from 4.7 pg/spot to 18.8 pg/spot. The results, as shown in FIG. 9, indicated that between 1.25 pg and 2.5 pg of applied antigen was detected above background. This was most favorably accomplished at lower capture antibody densities, as shown in FIG. 10.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” for “step” clause as specified in 35 U.S.C. § 112.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. An analyte detection device comprising:
a) a substrate; and
b) an array of detection spots on the substrate, each detection spot comprising an analyte sensor and a first binding ligand; wherein
i. the analyte sensor is bound to the substrate by the binding ligand;
ii. there are a plurality of analyte sensors for different analytes; and
iii. the same first binding ligand is used for two, or more than two different analyte sensors.

2. The analyte detection device of claim 1 wherein the binding ligand is immobilized directly to the substrate.

3. The analyte detection device of claim 1 further comprising a second binding ligand wherein the second binding ligand is used for two, or more than two different analyte sensors.

4. The analyte detection device of claim 1 wherein the substrate has pendant acyl fluoride functionalities and the detection spots are immobilized on the substrate by covalent bonding of the binding ligand to the pendant acyl fluoride functionalities.

5. The analyte detection device of claim 1 wherein the binding ligand and the analyte sensor are applied to the substrate in a predetermined pattern of substantially localized spots by printing.

6. The analyte detection device of claim 1 wherein the binding ligand is one of a protein, enzyme, carbohydrate, nucleic acid, oligonucleotide, polynucleotide, aptamer, hapten, drug, dye, small organic molecule, cell, cell fragment, receptor, cell surface binding agent, or an analog, mimic, conjugate, or composite thereof.

7. The analyte detection device of claim 1 wherein the binding ligand is one of Protein A, biotin, or streptavidin.

8. The analyte detection device of claim 1 wherein the analyte sensor is an antibody.

9. The analyte detection device of claim 1 wherein the analyte sensors each individually are selected from the group consisting of nucleic acids, proteins, carbohydrates, dyes, haptens, drugs, receptors, cell fragments or cells, their analogs, mimics, conjugates or composites thereof.

10. The detection device of claim 1 wherein the array of detection spots is comprised of a matrix of substantially localized spots.

11. The detection device of claim 1 wherein each detection spots in the array is at least about 10 microns in spot diameter.

12. The detection device of claim 1 wherein each detection spots in the array is about 75 microns to 150 microns in spot diameter.

13. The detection device of claim 1 wherein the array of detection spots is comprised of 100 to 400 spots.

14. A method for detecting a plurality of different analytes in a sample comprising:
a) selecting the analyte detection device of claim 1; and
b) placing an analytical sample onto the substrate substantially only on the detection spots.

15. The method of claim 14 further comprising:
c) washing the substrate to remove non-bound sample; and
d) placing a detection label onto the substrate substantially only on the detection spots.

16. The method of claim 14 further comprising the steps of:
e) washing non-bound detection label from the substrate; and
f) detecting bound detection label.

17. The method of claim 14 wherein the analytical sample is placed substantially only on the detection spots by printing.

18. The method of claim 14 wherein the detection label is placed substantially only on the detection spots by printing.

19. A method for detecting a plurality of different analytes in a sample comprising:
a) selecting an analyte detection device comprising:
i. a substrate; and
ii. an array of detection spots on the substrate, wherein each detection spot comprises an analyte sensor and a binding ligand, and wherein the analyte sensor is bound to the substrate by the binding ligand; and
b) printing more than one analytical sample substantially only on each detection spot.

20. A method for preparing an analyte detection device comprising placing a plurality of analyte sensors on an array of binding ligand spots, wherein each analyte sensors is for a different analyte, and wherein the same binding ligand is used for two, or more than two different analyte sensors, and wherein each analyte sensor is placed substantially only on each binding ligand spot by printing.

21. The method of claim 20 wherein the analyte sensor is one of streptavidin, Protein A, or biotin.

22. A method for detecting analytes in a sample comprising placing an analytical sample on an array of analyte sensor spots, wherein the array of analyte sensor spots is comprised of two or more than two different analyte sensor spots, and the analytical sample is placed substantially only on each analyte sensor spot by printing.

23. The method of claim 22 further comprising printing a detection label substantially only on each analyte sensor spot.

24. The method of claim 22 wherein at least one of the analyte sensor spots is an antibody.

25. A method for multi-step synthesis comprising:
a) selecting a substrate;
b) placing an array of binding ligand spots on the substrate; and
c) placing one or more than one synthetic reagents on the array of binding ligand spots, wherein there are a plurality of different reagents for different syntheses, and wherein the same binding ligand is used for two or more than two different syntheses, and wherein the synthetic reagents are placed substantially only on each binding ligand spot by printing.

26. The method of claim 25 wherein the synthetic reagent is a nucleotide or an amino acid derivative.
